(19) United States
(12) Patent Application Publication
(10) Pub. No.: US 2005/0125053 A1
(43) Pub. Date: Jun. 9, 2005

(54) Medical device having a tubular portion

(76) Inventors: Daniel Yachia, 46762 Herzliya (IL); Shay Galili, 69106 Tel Aviv (IL); Ronnie Levy, 44862 Zur Yigal (IL); Etty Plachinsky, Gush Tel-Mond (IL); Yaacov Kasavi, 44453 Kfar Sava (IL)

Correspondence Address: NATH & ASSOCIATES, 1030 15th STREET, NW, 6TH FLOOR, WASHINGTON, DC, 20005, US

(21) Appl. No.: 10/515,029

(22) PCT Filed: May 22, 2003
(86) PCT No.: PCT/IL03/00426

(30) Foreign Application Priority Data
May 23, 2002 (IL) 149828

(51) Int. Cl. A61F 2/06; A61M 5/00; A61M 25/00

(52) U.S. Cl. 623/1.15; 604/264

(57) ABSTRACT

A medical device having a tabular portion. In the wall of the tabular portion is a row of slots separated by portions of the wall referred to as “ribs”. The device my be folded by forming a longitudinal groove in the tubular portion separating two longitudinal lobes, with a row of alternating ribs and slots located in each lobe. Each rib in one lobe is then inserted into a slot in tie other lobe.

### FIELD OF THE INVENTION

This invention relates to medical devices and more specifically to such devices having a tubular potion such as a stent or a catheter.

### BACKGROUND OF THE INVENTION

Many medical devices that are inserted into the body have a tubular portion. For example, stents are tubular devices that are inserted into body ducts for preventing narrowing of the duct lumen, for tutoring a dilated lumen or for acting as a substrate for tissue growth. As another example, a catheter may have a tubular portion that may serve to transfer a fluid from outside the body to a body cavity, or for draining fluid from a body cavity.

The tubular portion of a medical device may have a fixed caliber in which it is both delivered and deployed. Alternatively, the tubular portion may be brought into an initial small caliber conformation in which it is inserted into the body and delivered to the site where it is to be deployed. Deployment of the device involves expanding the tubular portion to a final larger caliber. Thus, a stent may be brought into an initial small caliber conformation in which it is inserted into the body and delivered to the site where it is to be deployed, and then expanded.

Several stents are known in the art that are delivered to the site of their deployment in a small caliber conformation and then deployed in a large caliber conformation. European Patent No. 0382014 discloses a stent made of a flat sheet rolled as a scroll. U.S. Pat. No. 5,151,105 discloses collapsing the coils of a helical stent. U.S. Pat. No. 5,246,445 discloses tightly wrapping the coils of the stent around a catheter. WO 83/000997 discloses making a stent using curved metal wires interwoven into a mesh that is brought into a small caliber conformation by stretching the stent longitudinally. These stents are expanded after delivery either by inflating a balloon positioned in its lumen or by self expansion in the case of an elastic stent material when a constraining mechanism is released, or by a change in temperature in the case of a thermoexpandable stent material. Most stents tend to elongate when brought into the smaller caliber state. This limits the extent to which the caliber may be decreased before the length of the stent becomes too long and difficult to deliver. Stents that elongate when the caliber is decreased subsequently shorten when expanded during deployment. This shortening makes accurate positioning difficult.

European Patent 0221570 discloses an expandable stent that is a thin-walled tube made from a wire mesh. When a balloon is inflated in the lumen of the stent, a geometrical deformation of the mesh occurs as the stent expands radially. However, this stent may remain rigid, making it difficult to negotiate bends or tortuosities during delivery. Furthermore, the geometrical deformation during expansion may cause multiple weakenings in the wall of the stent thus increasing the possibility of spontaneous breakage.

U.S. Pat. No. 5,037,427 discloses a stent made from a two-way shape memory alloy. This stent has a transition temperature that is below body temperature in which it changes its diameter from a narrow diameter to a wide diameter. The stent is inserted into the body under a constant flow of cold fluid in order to maintain the stent in the narrow diameter during delivery. Once in the stent has been positioned in the desired location, the flow of the cold fluid is stopped and the stent then expands as it warms up to body temperature. When the stent is to be removed, a flow of cold fluid is again applied to the stent causing the stent to return to the narrow diameter conformation. The flow of cold fluid is maintained until the stent is removed from the body.

U.S. Pat. No. 6,042,605 discloses a flexible stent that may be folded longitudinally in order to bring the stent into a smaller caliber. The stent may be maintained in the folded, small caliber state by a guidewire that passes through loops formed along both sides of the longitudinal fold. After positioning in the body, the guidewire is removed, and the stent assumes its original large caliber conformation.

### SUMMARY OF THE INVENTION

The present invention provides a medical device having a tubular portion such as a stent or catheter. In accordance with the invention, the wall of the tubular portion contains at least two rows of slots. The slots may be transverse, parallel or oblique to the longitudinal axis of the tubular portion. Each pair of adjacent slots in a row is separated by a portion of the wall material referred to as a “rib”.

The tubular portion of the device may be folded into a small caliber, folded configuration by forming one or more longitudinal grooves in the wall of the tubular portion and inserting ribs in one row into slots in another row. The formation of a groove creates two longitudinal lobes in the wall of the tubular portion. In one method of folding the tubular portion, the longitudinal groove is formed so that the each lobe contains a row of slots, with a slot in one row being opposite a rib in the other row. In another method for folding the tubular portion, the longitudinal groove is formed so that one row of slots overlies another row of slots in the groove with each of one or more ribs in one row overlying a slot in the other row. In this case, each of one or more ribs in one or both of the rows is inserted into the juxtaposed slot in the other row. The tubular portion may then be maintained in the folded configuration by means of a rod extending through a lumen that is common to the two lobes.

The tubular portion of the device may be formed from a resiliently flexible material such as latex or silicone rubber. The tubular portion is in an unstrained state when in the unfolded configuration. The tubular portion may be maintained in the folded configuration by inserting it into a restraining sleeve. After positioning the device in the body, the restraining sleeve is removed and the tubular portion reverts to its initial unfolded and unconstrained configuration. Alternatively, the tubular portion may be maintained in the folded configuration by inserting a longitudinal rod or wire through ribs that have been inserted into slots. After deployment, the wire or rod is withdrawn so as to allow the tubular portion to regain its unfolded configuration. In yet another alternative, the tubular portion may be maintained in the folded configuration by coating the tubular portion with a thin solid coating. After deployment, the coating is mechanically or chemically degraded so as to allow the tubular portion to regain its unfolded configuration.

The tubular portion may also be made from a metal or a polymeric material that deforms into its plastic zone when folded. The tubular portion is then unfolded by inflating a balloon in the lumen of the tubular portion.

The tubular portion may also be formed from a super-elastic and/or shape memory alloy such as a nickel-titanium alloy or shape memory polymer. The alloy or polymer is trained to pass from the unfolded configuration to the folded configuration when subjected to a first process such as a temperature change, and to pass from the folded configuration to the unfolded configuration when subjected to a second process. The first process may be, for example, a temporary increase in temperature, and the second process may be a temporary decrease in temperature.

In its first aspect, the invention thus provides a medical device having at least a tubular portion, the tubular portion containing two or more slots separated by ribs.

In its second aspect, the invention provides a method for deploying in a body the medical device according to any one of the previous claims comprising:
(a) maintaining the device in the folded configuration
(b) positioning the device in the body;
(c) bringing the tubular portion into the unfolded configuration.

### BRIEF DESCRIPTION OF THE DRAWINGS

In order to understand the invention and to see how it may be carried out in practice, a preferred embodiment will now be described, by way of non-limiting exile only, with reference to the accompanying drawings, in which:

FIG. 1 shows a tubular portion of a medical device in accordance with one embodiment of the invention and a method for folding of the tubular portion from an unfolded configuration to a folded configuration;

FIG. 2 shows another method for folding the tubular portion of a medical device of the invention; and

FIG. 3 shows a tubular portion of a medical device in accordance with another embodiment of the invention and a method for holding it.

### DETAILED DESCRIPTION OF THE INVENTION

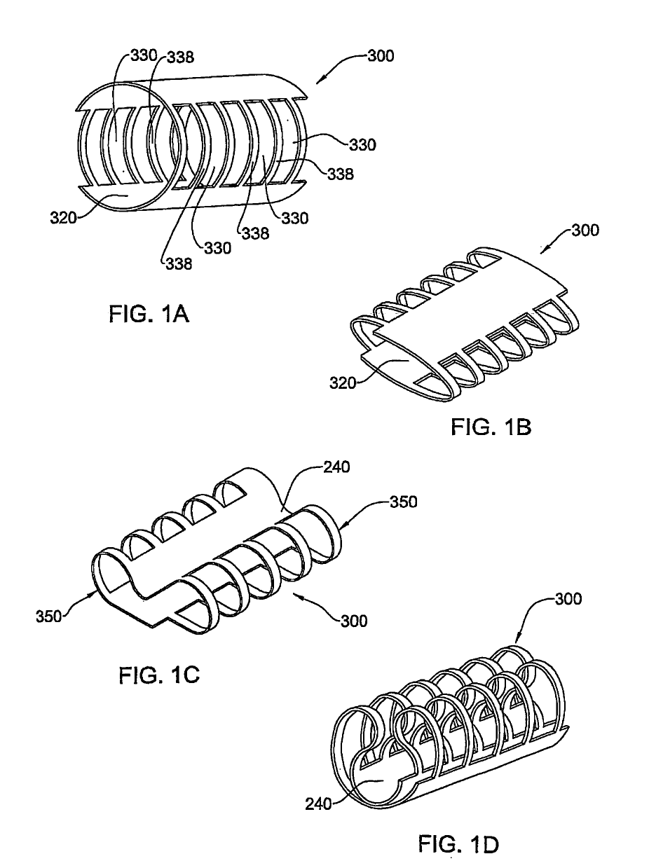

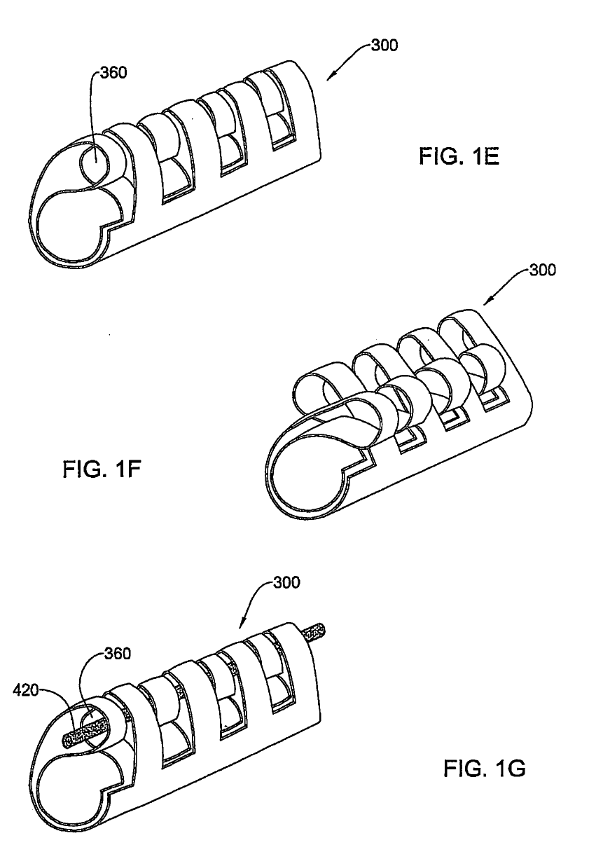

FIG. 1 shows a tubular portion generally indicated by 300 of a medical device, that may be, for example, a stent or catheter. The tubular portion 300 is a thin walled tube. The tubular portion 300 is shown in FIG. 1a in its unfolded configuration in which it is to be deployed in the body. The tubular portion 300 encloses a lumen 320. In the unfolded configuration shown in FIG. 1a, the lumen 320 has a circular cross-section. This is by way of example only, and the device of the invention may have any cross-sectional shape as required in any particular application. For example, a prostatic urethral stent may preferably have a triangular, oval, or hourglass shape. The tubular device may have a cross-sectional shape or size that varies along its length.

The tubular portion 300 has two rows of slots 330. This is by way of example only, and the tubular portion may have any number of rows or slots. Two adjacent slots 330 in a row are separated by a portion 338 of the tubular portion 300 referred to as a “rib ”. A slot in one row is in a cross-section with a rib in the other row. A slot in one row and a rib in another row that lie in the same cross-section are referred to herein as being “contiguous” with each other.

FIG. 1 shows several stages in a first process of bringing the tubular portion 300 into a folded configuration In FIG. 1b the tubular portion 300 is flattened, so as to decrease the cross-sectional area of the lumen 320. In FIG. 1c, a longitudinal groove 240 is introduced in the tubular portion 300. Formation of the groove 240 separates two longitudinal lobes 350 along the tubular portion 300. The groove is located between the two rows of slots so that each row of slots 330 is located in a different lobe 350. In FIG. 1d, the lobes 350 are brought near to each other. The ribs in each row are then inserted into contiguous slots in the other row. Inserting the slots 330 into the ribs 338 may be done in such a way so as to create a lumen 360 common to both lobes, as shown in FIG. 1e. Alternatively, a lumen common to both lobes is not formed as shown in FIG. 1f. The tubular portion may then be maintained in the folded configuration by means of a rod or wire 420 extending through the lumen 360 that is common to the two lobes as shown in FIG. 1f.

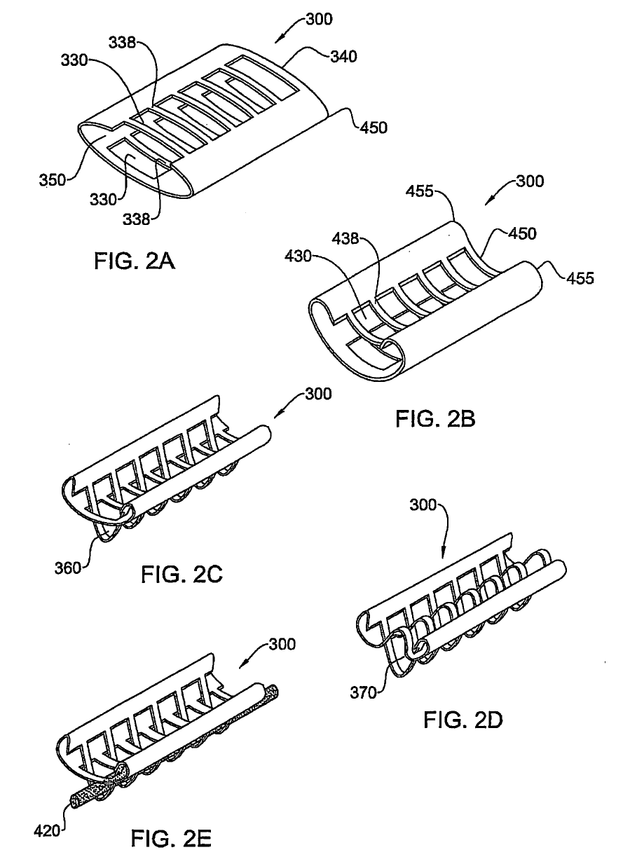

FIG. 2 shows several stages in a second process of bringing the tubular portion 300 into the folded configuration. In FIG. 2a the tubular portion 300 is flattened, so as to decrease the cross-sectional area of the lumen 320. In FIG. 2b, a longitudinal groove 450 is introduced in the tubular portion 300. Formation of the groove 450 separates two longitudinal lobes 455 along the tubular portion 300. In this method of folding, both rows of slots are located in the groove so that the two rows of slots are juxtaposed, with ribs in one row juxtaposed to slots in the other row. Each of one or more ribs in one of the two rows may then be inserted into a slot in the other lobe as shown in FIG. 2c. A lumen 360 is thus formed from the inserted ribs. Alternatively, as shown in FIG. 2d, ribs in both rows may be inserted into slots in the other row to form a lumen 360.

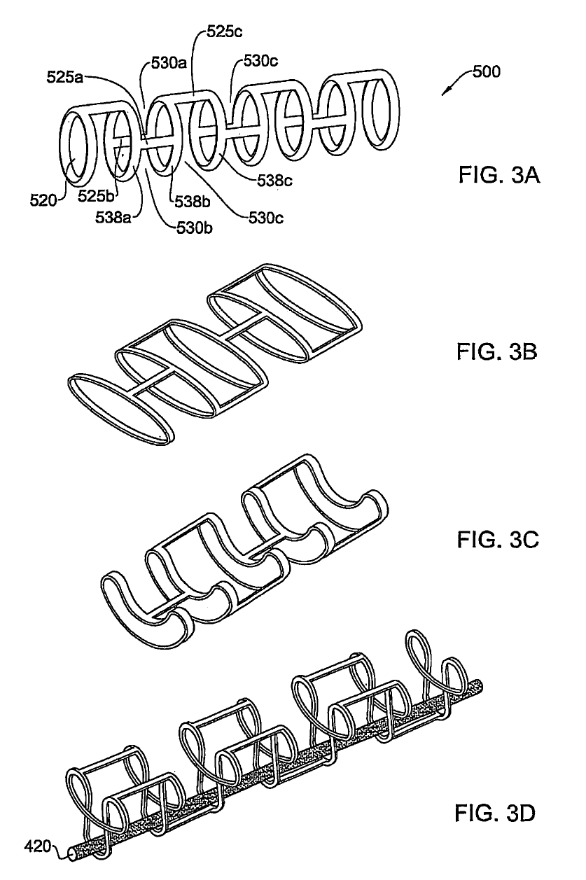

FIG. 3 shows a tubular portion generally indicated by 500 of a medical device, that may be, for example, a stent or catheter in accordance with another embodiment of the invention. The tubular portion 500 is shown in FIG. 3a in its unfolded configuration in which it is to be deployed in the body. The tubular portion 500 encloses a lumen 520. In the unfolded configuration shown in FIG. 3a, the lumen 520 has a circular cross-section. This is by way of example only, and the device of the invention may have any cross-sectional shape as required in any particular application. For example, a prostatic urethral stent may preferably have a triangular, oval, or hourglass shape. Tie tubular device may have a cross-sectional shape or size that varies along its length.

The tubular portion 500 has a plurality of ribs, for example, ribs 538a, 538b and 538c. The ribs 538 are joined together by bridges, for example, the bridges 525a, 525b and 525c. The bridges may be of any shape as required in a particular application. The number of bridges between adjacent ribs may vary, and in the embodiment shown in FIG. 3, alternates between 1 and 2. The ribs 538 are separated by slots, for example, the slots 530a, 530b and 530c . The number of slots 530 between adjacent ribs can also vary, and in the embodiment shown in FIG. 3 alternates between 1 and 2. Thus, for example, the ribs 538a and 538b are joined by two bridges 525a and 525b and two slots 530a and 530b. Ribs 538b and 538c are separated by the bridge 525c and the slot 530c.

FIG. 3 shows several stages in a first process of bringing the tubular portion 500 into a folded configuration. In FIG. 3b the tubular portion 500 is flattened, so as to decrease the cross-sectional area of the lumen 520. In FIG. 3c, a longitudinal groove 540 is introduced in the tubular portion 500. Formation of the groove 540 separates two longitudinal lobes 550 along the tubular portion 500. The groove is formed by pushing the row of single bridges (e.g. the row containing the bridges 525c) through the lumen 520 of the tubular portion 500. FIG. 3d shows the tubular portion in the folded configuration.

During insertion and delivery of the device in the body, the tubular portion is maintained in the folded configuration. When the tubular portion has been positioned in the body where it is to be deployed, it is made to return to the unfolded configuration. In the case of a tubular portion formed from an elastic material, the tubular portion may be maintained in the unfolded configuration by coating the tubular portion with a polymeric coating. For example, a 2:3 solution of silicone rubber and a solvent may be applied to the tubular portion 300 and allowed to cure. The solution may be applied by any known method such as brushing, dipping or immersion. After positioning in the body, the coating is degraded either mechanically or chemically to allow expansion of the tubular portion. Alternatively, the tubular portion may be maintained in the folded configuration by inserting a wire or rod 420 into a lumen of the folded tubular portion as shown in FIGS. 1g, 2e and 3d. After the device has been positioned in the body, the wire or rod 420 is withdrawn and the tubular portion returns to its unfolded share.

If the tubular portion is made from a material that deforms into its plastic zone when brought into the folded configuration, the tubular portion can brought back to the unfolded configuration by introducing a balloon into the lumen of the folded configuration, as shown in FIG. 4a, and inflating the balloon, as shown in FIG. 4b (not shown).

The tubular portion may also be made from a one or two way shape memory alloy such as Nitinol. The shape memory alloy is processed by methods known in the art so that when the shape memory alloy is in its superelastic or austenite state, the tubular portion 300 is in the unfolded configuration. The shape memory alloy is further processed so that when it is in its soft or austenite state, the tubular portion 300 is in the folded configuration. The tubular portion is brought into the folded configuration by causing it to undergo a transition from the martensite state to the austenite state. This may be accomplished, for example, by temporarily cooling the tubular portion to a temperature at which this transition occurs. After positioning in the body, the tubular portion is brought into the unfolded configuration by causing it to undergo a transition from the austenite state to the martensite state. This may be accomplished, for example, by temporarily heating the tubular portion to a temperature at which this transition occurs.

The shape memory alloy preferably has a transition temperature range from the martensite state to the austenite state and a transition temperature range from the state that are near body temperature (around 37° C.). This allows the shape memory alloy to undergo a transformation from one state to the other at temperatures that essentially do not harm the surrounding body tissues.

1. A medical device having at least a tubular portion, the tubular portion containing two or more slots separated by ribs.

2. The device according to claim 1 wherein the slots are arranged in two or more rows with a slot in one row being contiguous with a rib in another row.

3. The device according to claim 2 wherein the tubular portion has been brought from an unfolded configuration into a folded configuration by a method comprising:
(a) forming one or more longitudinal or helical grooves in the tubular portion separating two or more longitudinal lobes so that each row of slots is located in a different lobe; and
(b) inserting each of one or more ribs in one lobe into a slot in another lobe.

4. The device according to claim 2 wherein the tubular portion has been brought from an unfolded configuration into a folded configuration by a method comprising:
(a) juxtaposing the two rows of slots; and
(b) inserting one or more ribs in one row into a slot in the other row.

5. The device according claim 1 wherein the tubular portion is formed from an elastic material.

6. The device according to claim 5 wherein the tubular portion is maintained in the folded configuration by inserting the tubular portion in the folded configuration into a restraining sleeve.

7. The device according to claim 3 wherein the tubular portion is maintained in the folded configuration by applying a polymeric coating to the tubular portion and allowing the coating to cure.

8. The device according to claim 7 wherein the coating is mechanically or chemically degradable.

9. The device according to claim 8 wherein the coating is applied by spraying, dipping, or brushing.

10. The device according to claim 3 wherein the tubular portion deforms into its plastic zone when brought into the folded configuration.

11. The device according to claim 3 wherein the tubular portion is formed from a shape memory material, the tubular portion passing into the folded configuration when the shape memory material undergoes a transition from the martensite state to the austenite state.

12. The device according to claim 11 wherein the shape memory alloy is a nickel-titanium alloy.

13. The device according to claim 12 wherein the shape memory material is a shape memory polymer.

14. The device according to claim 1 wherein a lumen is formed by the inserted ribs.

15. The device according to claim 14 further comprising a rod inserted into the lumen common so as to maintain the device in the folded configuration.

16. The device according to claim 1 wherein the device is a stent or a catheter.

17. The device according to claim 1 wherein the tubular portion has a circular, triangular or hourglass shape.

18. The device according to claim 1 which the tubular portion has a cross-sectional size or shape that varies along the length of the tubular portion.

19. A method for deploying in a body the medical device according to claim 1 comprising:
(a) maintaining the device in the folded configuration
(b) positioning the device in the body;
(c) bringing the tubular portion into the unfolded configuration.

20. The method according to claim 19 wherein the tubular portion is formed from an elastic material, and wherein a restraint is applied to the tubular portion in the folded configuration so as to maintain the tubular portion in the folded configuration and wherein bringing the device into the unfolded configuration involves removing the restraint.

21. The method according to claim 20 wherein the restraint is applied by inserting the tubular portion in the folded configuration into a restraining sleeve, and wherein removing the restraining sleeve.

22. The method according to claim 19 wherein the restraint is applied by applying a polymeric suspension to the tubular portion in the folded configuration and allowing the suspension to cure so as to form a coating to the tubular portion and wherein bringing the tubular portion into the unfolded configuration involves degrading at least a portion of the coating.

23. The method according to claim 22 wherein the polymeric coating is applied by spraying, dipping or brushing.

24. The method according to claim 19 wherein the tubular portion is made from a shape-memory material that has been trained to pass from the unfolded configuration to the folded configuration when subjected to a first process and to pass from the folded configuration to the unfolded configuration when subjected to a second process, and wherein bringing the tubular portion from the unfolded configuration to the folded configuration involves applying the first process, and wherein bringing the tubular portion from the folded configuration to the unfolded configuration involves applying the second process.
